(19) United States
(12) Patent Application Publication
(10) Pub. No.: US 2022/0087648 A1
(43) Pub. Date: Mar. 24, 2022

(54) 3-D Endocavity Ultrasound Probe with a Needle Guide

(71) Applicant: B-K Medical ApS, Herlev (DK)

(72) Inventors: Fredrik Gran, Limhamn (SE); Henrik Jensen, Nordhavn (DK); Bo Martins, Rodovre (DK); Bradley Nelson, Centre Hall, PA (US)

(73) Assignee: B-K Medical ApS, Herlev (DK)

(21) Appl. No.: 17/025,394

(22) Filed: Sep. 18, 2020

(51) Int. Cl. A61B 8/08; A61B 8/12; A61B 8/00; A61B 8/14

(52) U.S. Cl. CPC A61B 8/483; A61B 8/12; A61B 8/445; A61B 8/145; A61B 8/4488; A61B 8/4477; A61B 8/4461

(57) ABSTRACT

A 3-D endocavity ultrasound probe includes an instrument holder having a channel configured to guide an instrument. The 3-D endocavity ultrasound probe further includes an elongate shaft with a center line, a first side, a second opposing side with a recess configured to receive the instrument holder over the center line, and a drive system disposed in the first side and not over the center line. The 3-D endocavity ultrasound probe further includes a probe head disposed at an end of the shaft. The probe head includes a rotatable support and a transducer array coupled to the rotatable support. The drive system is configured to rotate the rotatable transducer array support thereby rotating the transducer array. The channel extends along the center line thereby providing an instrument path in-plane with a sagittal plane of the elongate shaft.

### TECHNICAL FIELD

The following generally relates to ultrasound imaging and more particularly to a three-dimensional (3-D) endocavity ultrasound probe with a needle guide.

### BACKGROUND

Ultrasound imaging has provided useful information about the interior characteristics of an object or subject under examination. For example, ultrasound-guided prostate biopsy has been used to assist with removing a tissue sample(s) from a suspect area of the prostate, e.g., to rule out/diagnose cancer. With a transrectal prostate biopsy procedure, a 2-D endocavity ultrasound imaging probe with a biopsy needle guide installed in the probe shaft is inserted into the rectum via the anus. A biopsy needle is advanced to the prostate using the biopsy needle guide to guide the biopsy needle along a center-line of the shaft and in-plane in the sagittal plane of the transducer array through the rectal wall to the prostate based on images.

FIG. 1 shows an example of a 2-D endocavity ultrasound imaging probe during a transrectal prostate biopsy procedure. The probe 102 includes a handle 104, an elongate shaft 106 with a biopsy guide 108 installed therein, and a probe head 110, which houses a transducer array configured to produce a sagittal scan plane. The biopsy guide 108 includes needle ports 112 and 114 and a needle channel 116 inside of the elongate shaft 106 between the ports 112 and 114 that guides a biopsy needle 118. FIG. 2 shows the biopsy guide 108 disengaged from the elongate shaft 106. When engaged, the needle channel 116 extends along a center-line 202 of the elongate shaft 106 and transducer array 204 and guides the biopsy needle 118 in-plane with the sagittal scan plane of the transducer array 204. This allows the biopsy needle 118 to be imaged in the sagittal scan plane of the transducer array 204.

State-of-the-art 3-D endocavity ultrasound imaging probes with rotating/wobbling transducer arrays in the probe head or shaft cannot be used with a biopsy guide configured as in FIG. 1 because the drive system for controlling the rotating/wobbling is inside of the shaft, which inhibits advancement of the biopsy needle through a biopsy guide installed in the shaft and along a center-line and in the sagittal plane of the shaft and hence the sagittal scan plane of the transducer array. To guide advancing the biopsy needle otherwise (e.g., off-center) requires multiple images at different rotations to image the biopsy needle, which adds time and complexity. FIG. 3 shows a perspective view of a 3-D endocavity ultrasound imaging probe 302 with a transducer array 304 in a probe head 306 and configured to wobble about an axis 308 and a drive system 310 for controlling the wobbling inside of a shaft 312.

Transrectal biopsies have been performed where the entire prostate is systematically, but randomly sampled, leading to discomfort to the patient. Another technique is fusion biopsy. With this technique, a real-time 2-D ultrasound image is fused with pre-procedure 3-D volumetric data (e.g., magnetic resonance (MR) or computerized tomography (CT) data) in which the prostate has been segmented. This allows for targeting the biopsy to regions where legions have been identified in the 3-D volumetric data, potentially resulting in more accurate diagnosis. This technique requires identifying common landmarks in the 2-D image and the 3-D volume to register them together. Unfortunately, the fusion biopsy process can be time consuming and registration process prone to errors and inaccuracy.

In view of at least the foregoing, there is an unresolved need for an improved endocavity ultrasound probe with a biopsy needle guide that is configured for transrectal prostate biopsy procedures.

### SUMMARY

Aspects of the application address the above matters, and others.

In one aspect, a 3-D endocavity ultrasound probe includes an instrument holder having a channel configured to guide an instrument. The 3-D endocavity ultrasound probe further includes an elongate shaft with a center line, a first side, a second opposing side with a recess configured to receive the instrument holder over the center line, and a drive system disposed in the first side and not over the center line. The 3-D endocavity ultrasound probe further includes a probe head disposed at an end of the shaft. The probe head includes a rotatable support and a transducer array coupled to the rotatable support. The drive system is configured to rotate the rotatable transducer array support thereby rotating the transducer array. The channel extends along the center line thereby providing an instrument path in-plane with a sagittal plane of the elongate shaft.

In another aspect, a 3-D endocavity ultrasound probe including a transducer array. The 3-D endocavity ultrasound probe further includes a shaft housing a drive system configured to rotate the transducer array. The 3-D endocavity ultrasound probe further includes. The drive system, including an electrical interconnect, is disposed off-center of a center-line of the shaft. The channel is disposed along the center-line of the shaft.

In yet another aspect, a method includes rotating a transducer array of an endocavity ultrasound probe. The endocavity ultrasound probe further includes a shaft housing a drive system configured to rotate the transducer array and an instrument holder with a channel configured to guide an instrument. The method further includes advancing an instrument, via the guide, in a sagittal plane of the shaft. The method further includes acquiring 3-D ultrasound data with the rotating transducer array.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings, in which like references indicate similar elements and in which:

FIG. 1 illustrates a prior art 2-D endocavity ultrasound imaging probe with a biopsy needle guide configured for transrectal biopsy procedures;

FIG. 2 illustrates the biopsy needle guide disengaged from a shaft of the probe of FIG. 1;

FIG. 3 illustrates a prior art 3-D endocavity ultrasound imaging probe with a drive system for rotating/wobbling the transducer array located in the shaft;

FIG. 4 illustrates a perspective view of a 3-D endocavity ultrasound imaging probe with a drive system for rotating/wobbling the transducer array located off-center in a shaft and out of the way of a center-line and sagittal plane of the shaft; in accordance with an embodiment(s) herein;

FIG. 5 illustrates a top down view of the 3-D endocavity ultrasound imaging probe of FIG. 4, showing a recess in the shaft for an instrument guide and the instrument guide disengaged therefrom, in accordance with an embodiment(s) herein;

FIG. 6 illustrates a side view of the 3-D endocavity ultrasound imaging probe of FIG. 4, in accordance with an embodiment(s) herein;

FIG. 7 illustrates a perspective view of a variation of the 3-D endocavity ultrasound imaging probe illustrated in FIG. 1; in accordance with an embodiment(s) herein;

FIG. 8 illustrates a perspective view of another variation of the 3-D endocavity ultrasound imaging probe illustrated in FIG. 1; in accordance with an embodiment(s) herein;

FIG. 9 illustrates an example ultrasound imaging system including a probe described herein; in accordance with an embodiment(s) herein;

FIG. 10 illustrates an example method, in accordance with an embodiment(s) herein.

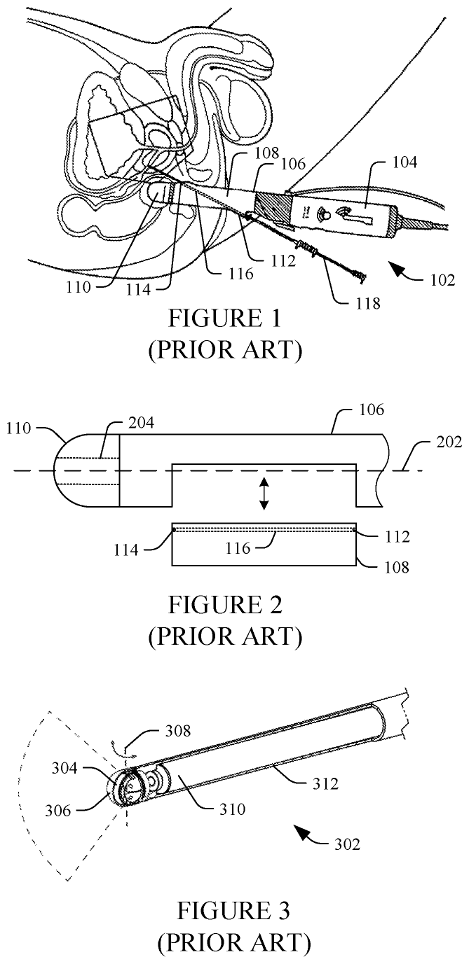

### DETAILED DESCRIPTION

The following describes a 3-D ultrasound endocavity probe(s) with a rotating transducer array and an instrument guide configured for transrectal prostate biopsy procedures. As described in greater detail below, the drive system for the rotating transducer array is inside of the shaft and offset from a center-line of the shaft and does not physically interfere with advancing an instrument inside of the shaft along the center-line and sagittal plane of the shaft and in-plane with a sagittal scan plane of a transducer array. In one instance, this allows for acquiring real-time 3-D ultrasound data that can efficiently and accurately fused with pre-procedure 3-D data, e.g., for an ultrasound-guided transrectal prostate biopsy procedure, etc.

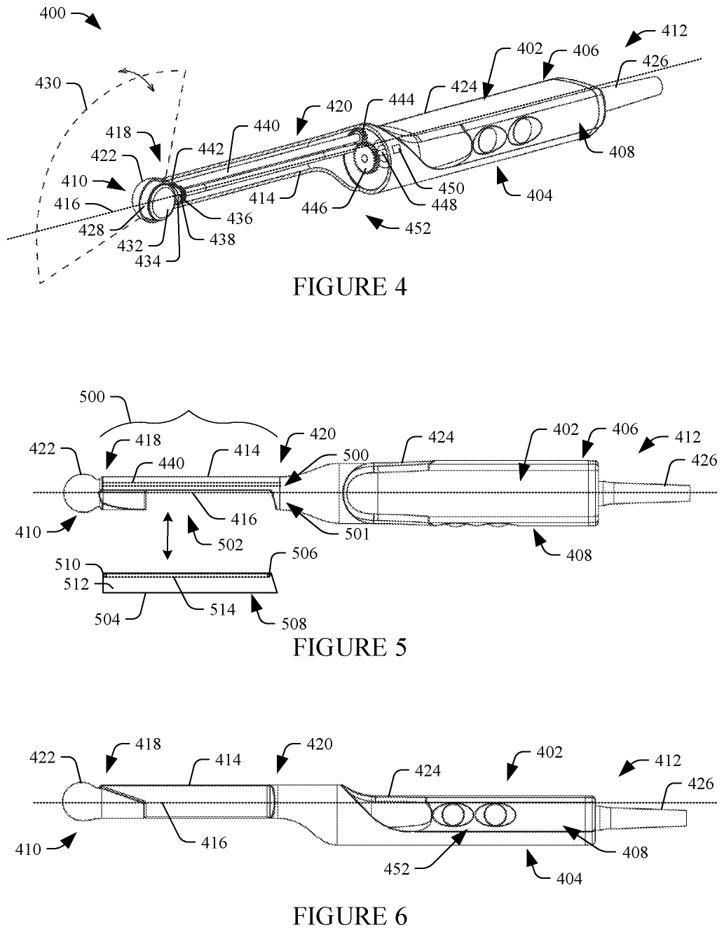

FIGS. 4-6 illustrate an example 3-D ultrasound endocavity probe 400. FIG. 4 illustrates a partially cross-sectioned perspective view, FIG. 5 illustrates a top down view, and FIG. 6 illustrates a side view. The 3-D ultrasound endocavity probe 402 generally has a top 402, a bottom 404, a first side 406, a second side 408 that opposes the first side 406, a front 410 and a back 412.

The probe 402 includes an elongate shaft 414 having a long central axis (center-line) 416, a first end region 418 of the long axis 416, and a second opposing end region 420 of the long axis 416. A probe head 422 is located at the first end region 418, and a handle 424 with controls is located at the second end region 420. In the illustrated embodiment, a cable 426 routes signals to and from the probe 402. In another embodiment the probe 402 alternatively, or additionally, includes a wireless communications interface for routing signals to and from the probe 402.

With reference to FIG. 5, the elongate shaft 414 includes a first side 500 and a second side 501 with a recess 502 configured to removably receive an instrument guide 504. A securing device such as a clamp, etc. can be used to secure the instrument guide 504 in the recess 502. The instrument guide 504 includes a first port 506 located at a bottom 508 of the guide 504 and a second port 510 located at a top 510 of the guide 504. A channel 514 extends diagonally in the guide 504 from the first port 506 to the second port 510, similar to FIG. 1. Non-limiting examples of instrument guides can be found in U.S. Pat. No. 6,443,902 B1, which is incorporated herein in its entirety by reference.

When the instrument guide 504 is engaged in the recess 502 of the elongate shaft 414, the channel 510 extends along the long central axis 416. As such, the instrument guide 504 can be used to advance an instrument along the center-line and sagittal plane of the elongate shaft 414 and transducer array. An example of the instrument guide 504 includes a biopsy needle guide, and an example of the instrument includes a biopsy needle. Other devices and guides for the other devices are contemplated herein. Although FIG. 5 shows only a single needle channel with a fixed trajectory/path, in another embodiment the instrument guide 504 includes only a single needle channel that may be articulated to different trajectory/path and/or more than one channel, e.g., configured for a different trajectory/path.

In FIG. 4, portions of the probe head 422, the elongate shaft 414, and the handle 424 are removed or shown invisible for explanatory purposes to describe components housed therein. With reference to FIG. 4, the probe head 422 houses a transducer array 428. In the illustrated embodiment, the transducer array 428 includes a curved (e.g., convex) array located on the long axis 416 and configured to provide a sagittal scan plane 430 with respect to the shaft 414. The illustrated transducer array 428 is a circular arc with a curvature of radius R.

In the illustrated example, the circular arc of the transducer array 428 subtends an angle greater than ninety-degrees. As such, the scan plane 430 can extend from below the long axis 416 to beyond perpendicular to the long axis 416, as illustrated in FIG. 4. This also allows for producing scan planes over a smaller angle, including an end fire scan plane and/or other scan plane. In another embodiment, the circular arc of the transducer array 428 subtends an angle less than that shown in FIG. 4. In another embodiment, the circular arc of the transducer array 428 subtends an angle greater than that shown in FIG. 4.

The transducer array 428 includes a 1-D or 2-D array of transducer elements. The one or more transducer elements include a piezoelectric, a capacitive micromachined ultrasonic transducer (cMUT), a thick film print, a composite and/or other type of transducer material. The one or more transducer elements are configured to convert an excitation electrical pulse into an ultrasound pressure field and convert a received ultrasound pressure field (an echo) into electrical (e.g., a radio frequency (RF)) signal.

The curved transducer array 428 is disposed on a curved outer surface (not visible) of a support 432. In one non-limiting instance, the support 432 is a spherical segment. The support 432 is rotatably coupled to a bearing 434 in the head 422 and/or the shaft 414. A circular toothed gear 436 is disposed around a protrusion 438 of the support 432. Generally, a center of the gear 436 is along the long central axis 416. The shaft 414 further includes a drive shaft 440. With reference to FIGS. 4 and 5, the drive shaft 440 is located off-center with respect to the long central axis 416 and inside of the shaft 414 in the first side 500 next to the recess 502. In this location, the drive shaft 440 does not physically interfere with the guide 504.

With reference to FIG. 4, the drive shaft 440 includes a toothed gear 442 at a first end with teeth that are configured to engage teeth of the toothed gear 436. The drive shaft 440 extends into the handle 424 and includes a second toothed gear 444 at an opposing end. The handle 424 include toothed gear 446 with teeth that are configured to engage teeth of the second toothed gear 444. The toothed gear 446 is coupled to a rod 448 that is coupled to a motor 450. Components 436 and 440-450 are referred to herein as a drive system 452.

It is to be understood that the illustrated drive system 452 is for explanatory purposes and other drive systems (e.g., a different gear based system, a belt drive system, etc.) are contemplated herein.

In general, when the motor 450 turns the rod 448, the toothed gear 446 turns the toothed gear 444 and hence the drive shaft 440 and the toothed gear 442, which turns the turns the toothed gear 436 and hence the support 432 and transducer array 428 supported thereby, which rotates the image plane 430 from the sagittal plane of the probe 402, and data can be acquired at different angular positions, with respect to the sagittal plane of the shaft 414. Images perpendicular to the shaft 414, images transverse to the shaft 414, and/or a 3-D volume can be created with the acquired data.

Variations are contemplated.

In a variation, the 3-D ultrasound endocavity probe 402 includes an optical and/or electromagnetic sensor configured to produce information that can be used to track the probe 402 and/or instrument relative to the patient.

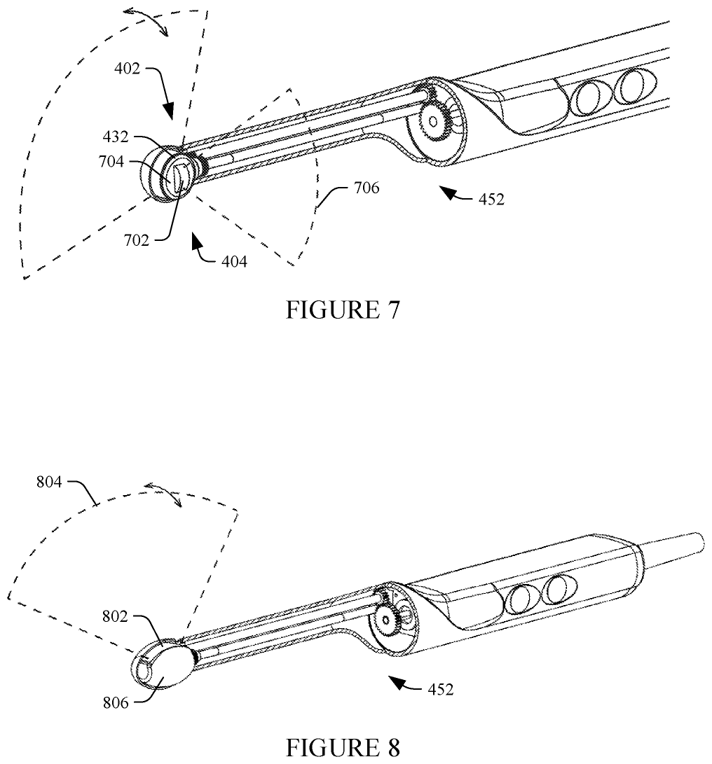

FIG. 7 illustrates a variation in which the 3-D ultrasound endocavity probe 402 further includes a second curved transducer array 702. The second curved transducer array 702 is disposed on a flat side 704 of the support 432, transverse to the curved transducer array 428, and extending between the top 402 and the bottom 404. The second curved transducer array 702 is configured to produce a transverse image plane 706.

Rotating the support 432 rotates the curved transducer array 428 and the second curved transducer array 702, and the image planes created by the curved transducer array 428 and the second curved transducer array 702 intersect. In the preferred embodiment, the second curved transducer array 702 is oriented perpendicular to shaft 414 and its image plane also intersects the center of the curved transducer array 428. A full transverse image can be constructed from a composite of many individual lines captured as the second curved transducer array 702 rotates. In one instance, this configuration allows for more control of image line formation for creation of a transverse image resulting in better image quality.

FIG. 8 illustrates a variation in which the 3-D ultrasound endocavity probe 402 includes a curved transducer array 802 with a radius of curvature R′, which is greater than R of the curved transducer array 428, configured to produce a scan plane 804, and ad support 806 rotatably coupled to the drive system 452.

In a variation, the 3-D ultrasound endocavity probe 402 of FIG. 8 includes a second curved transducer array similar to the second curved transducer array 702 illustrated in FIG. 7.

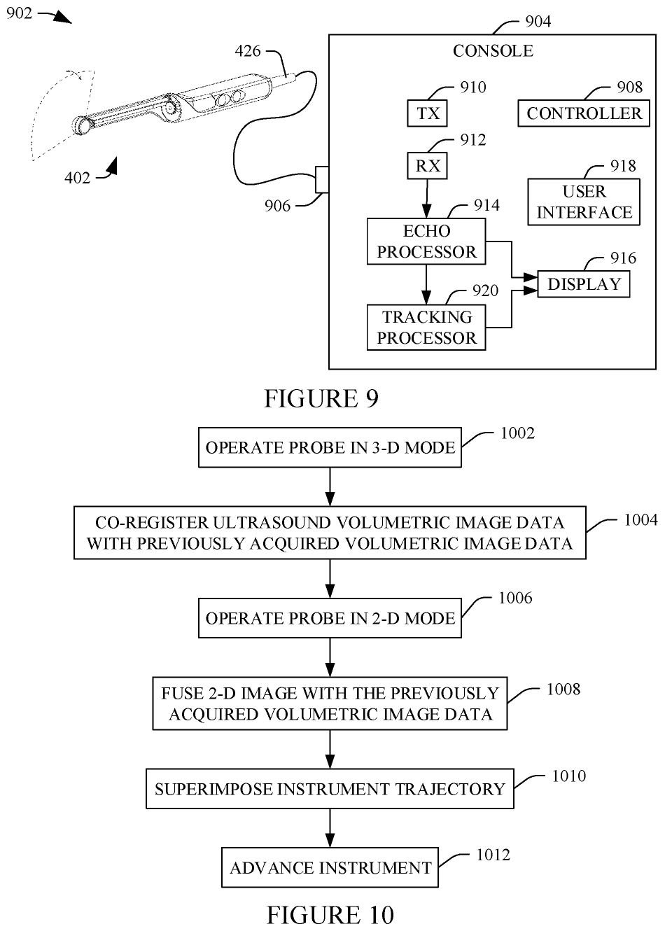

FIG. 9 illustrates an example imaging system 902 such as an ultrasound imaging system/scanner. The imaging system 902 includes the 3-D ultrasound endocavity probe 402 and a console 904. The console 904 includes an interface 906 configured to communicate with the communications interface 426 of the probe 402. In the illustrated embodiment, the interface 906 is an electromechanical connector configured to engage a complementary connector of the cable 426.

The console 904 further includes a controller 908 configured to control one or more of the components therein, the transducer arrays 428, 802 and/or 702, and the drive system 452. In one instance, when scanning with only the sagittal array 428 or 802, the rotatable support 432 or 806 is rotated at a low speed, such as at a non-limiting example of rotating plus and minus 105 degrees from the sagittal position at 1 Hz to capture a 3-D volume image dataset of the prostate. In another instance a 2-D sagittal and a 2-D transverse image may be displayed simultaneously by the ultrasound imaging system where the rotatable support 432 or 806 is rotated at a higher speed, such as at a non-limiting example of rotating plus and minus 105 degrees from the sagittal position at 10 Hz to create a composite transverse array image and capture a true sagittal array image at a center position aligned with the sagittal plane of the probe to visualize the relevant anatomy and instrument. In another instance the sagittal array 428 or 802 is statically positioned at a center position aligned with the sagittal plane of the probe to visualize the relevant anatomy and instrument.

The console 904 includes transmit circuitry (TX) 906 configured to generate the excitation electrical pulses and receive circuitry (RX) 908 configured to process the RF signals, e.g., amplify, digitize, and/or otherwise process the RF signals. The console 904 includes further an echo processor 914 configured to process the signal from the receive circuitry 908. For example, in one instance the echo processor 914 is configured to beamform (e.g., delay-and-sum) the signal to construct a scanplane of scanlines of data. The echo processor 914 can process data from 1-D and/or 2-D probes for 2-D, 3-D and/or 4-D applications. The echo processor 914 can be implemented by a hardware processor such as a central processing unit (CPU), a graphics processing unit (GPU), a microprocessor, etc.

The console 904 further includes a display 916 configured to display images generated by the echo processor 914. The console 904 further includes a user interface 918, which includes one or more input devices (e.g., a button, a touch pad, a touch screen, etc.) and one or more output devices (e.g., a display screen, a speaker, etc.). The controller 908 is configured to control one or more of the transmit circuitry 910, the receive circuitry 912, the echo processor 914, the display 916, the user interface 918, and/or one or more other components of the imaging system 902.

The illustrated embodiment further includes a tracking processor 920. In one instance, the tracking processor 920 is configured to fuse images generated by the echo processor 914 (e.g., 2-D, 3-D, etc.) with a previously generated 3-D volume (e.g., MR, CT, US, etc.). In one example, the fusion of the image generated by the echo processor 914 and the previously generated 3-D volume is achieved using information from an internal and/or external tracking device(s) (e.g., an optical and/or electromagnetic sensor) of the probe 402 and/or instrument, where the tracking device(s) tracks a spatial location of the probe 402 relative to the instrument.

With optical tracking, fiducial targets are placed on both the probe 402 and a needle of the biopsy instrument. The tracking processor 920 includes an optical device such as a video camera that records the spatial orientation of the optical elements to determine location and orientation. With electromagnetic tracking, tracking coils are included with both the probe 402 and a needle of the biopsy instrument. The tracking processor 920 measures a magnetic field strength of the coils, which depends on a distance and direction of the coils to the tracking processor 920, and the strength and direction is used to determine location and orientation.

Suitable tracking is discussed in Birkfellner et al., “Tracking Devices,” In: Peters T., Cleary K. (eds) Image-Guided Interventions. Springer, Boston, Mass., 2008. Suitable tracking systems are described in application publication number US 2010/0298712 A1, filed Feb. 10, 2010, and entitled “Ultrasound Systems Incorporating Position Sensors and Associated Method,” which is incorporated herein by reference in its entirety. Other approaches are also contemplated herein.

The tracking processor 920 utilizes the tracking signal/tracking data to register spatial coordinate systems of the probe 402 and the instrument and identify a cross-sectional plane in the 3-D ultrasound data that shows the instrument and its trajectory, and this image is displayed via the display 916. Where a pre-procedure scan (e.g., MRI, CT, etc.) is available and a target is located in the resulting 3-D data, the tracking processor 920 superimposes and registers the image generated by the echo processor 914 over the previously generated 3-D volume and selects and displays a plane that shows the instrument, its trajectory and the target.

In another embodiment, this is achieved through image based tracking. For instance, in one example this is achieved with “live segmentation and live alignment between the live images (e.g., 2-D, 3-D, etc.) generated by the echo processor 914 and the previously generated 3-D volume. An example of the later is described in PD09018, application Ser. No. 17/024,954, entitled “Image Fusion-Based Tracking without a Tracking Sensor,” filed on Sep. 18, 2020, and assigned to BK Medical ApS, which is incorporated herein by reference in its entirety.

FIG. 10 illustrates a method, in accordance with an embodiment(s) herein.

At 1002, the 3-D ultrasound endocavity probe 402 is operated in 3-D mode to capture volumetric image data, as described herein and/or otherwise.

At 1004, the volumetric image data is registered with previously acquired volumetric image data, as described herein and/or otherwise.

At 1006, the 3-D ultrasound endocavity probe 402 is operated in 2-D mode to capture a live 2-D image, as described herein and/or otherwise.

At 1008, the live 2-D image is fused with the previously acquired volumetric image data, as described herein and/or otherwise.

At 1010, a current trajectory of an interventional instrument to a target is superimposed on the fused image, as described herein and/or otherwise.

At 1012, the interventional instrument is advanced, as described herein and/or otherwise.

Acts 1008 to 1012 are repeated, e.g., at least until the interventional instrument is at the target.

In one instance, the 3-D data is utilized for tracking, e.g., in connection with a transrectal prostate biopsy procedure, as described herein and/or otherwise.

The above may be implemented at least in part by way of computer readable instructions, encoded or embedded on computer readable storage medium (which excludes transitory medium), which, when executed by a computer processor(s) (e.g., central processing unit (CPU), microprocessor, etc.), cause the processor(s) to carry out acts described herein. Additionally, or alternatively, at least one of the computer readable instructions is carried by a signal, carrier wave or other transitory medium (which is not computer readable storage medium).

The application has been described with reference to various embodiments. Modifications and alterations will occur to others upon reading the application. It is intended that the invention be construed as including all such modifications and alterations, including insofar as they come within the scope of the appended claims and the equivalents thereof.

What is claimed is:

1. A 3-D endocavity ultrasound probe, comprising:
an instrument holder, including:
a channel configured to guide an instrument;
an elongate shaft, including:
a center line;
a first side;
a second opposing side with a recess configured to receive the instrument holder over the center line; and
a drive system, including an electrical interconnect, disposed in the first side and not over the center line;
a probe head disposed at an end of the shaft, wherein the probe head includes:
a rotatable support; and
a transducer array coupled to the rotatable support;
wherein the drive system is configured to rotate the rotatable transducer array support thereby rotating the transducer array, and the channel extends along the center line thereby providing an instrument path in-plane with a sagittal plane of the elongate shaft.

2. The 3-D endocavity ultrasound probe of claim 1, wherein the transducer array includes only a single array disposed in a direction along the center line.

3. The 3-D endocavity ultrasound probe of claim 2, wherein rotatable support has a curved surface, the transducer array is curved, and the transducer array is affixed to the curved surface.

4. The 3-D endocavity ultrasound probe of claim 3, wherein rotatable support is a spherical segment.

5. The 3-D endocavity ultrasound probe of claim 1, wherein the transducer array includes at least a first array and a second array.

6. The 3-D endocavity ultrasound probe of claim 5, wherein the first array is disposed in a first direction along the center line.

7. The 3-D endocavity ultrasound probe of claim 6, wherein the rotatable support has a curved surface, the first array is curved, and the first array is affixed to the curved surface.

8. The 3-D endocavity ultrasound probe of claim 7, wherein the second array is disposed in a second direction that is transverse to the center line.

9. The 3-D endocavity ultrasound probe of claim 8, wherein the rotatable support further includes a flat side; and the second array is disposed is disposed on the flat side.

10. The 3-D endocavity ultrasound probe of claim 7, wherein the rotatable support is a spherical segment.

11. The 3-D endocavity ultrasound probe of claim 10, wherein the second array is disposed in a second direction that is transverse to the center line.

12. The 3-D endocavity ultrasound probe of claim 11, wherein the rotatable support further includes a flat side; and the second array is disposed is disposed on the flat side.

13. The 3-D endocavity ultrasound probe of claim 1, wherein the drive system includes: a shaft with a first end and a second end and a first gear disposed on the first end; and, wherein the rotatable support includes: a second gear, wherein the first gear is configured to engage the second gear to rotate the rotatable support.

14. The 3-D endocavity ultrasound probe of claim 13, further comprising:
a handle, wherein the handle includes:
a third gear; and
a motor configured to rotate the third gear, and wherein the shaft further includes:
a fourth gear on the second end,
wherein the third gear is configured to engage the fourth gear to rotate the shaft.

15. A 3-D endocavity ultrasound probe, comprising:
a transducer array;
a shaft housing a drive system configured to rotate the transducer array; and
an instrument holder with a channel configured to guide an instrument,
wherein the drive system is disposed off-center of a center-line of the shaft, and the channel is disposed along the center-line of the shaft.

16. The 3-D endocavity ultrasound probe of claim 15, wherein the transducer array includes a single transducer array disposed along the center-line of the shaft.

17. The 3-D endocavity ultrasound probe of claim 15, wherein the transducer array includes a first transducer array disposed along the center-line of the shaft and a second transducer array disposed transverse to the center-line of the shaft.

18. A method, comprising:
rotating a transducer array of an endocavity ultrasound probe, wherein the endocavity ultrasound probe further includes a shaft housing a drive system configured to rotate the transducer array and an instrument holder with a channel configured to guide an instrument;
advancing an instrument, via the guide, in a sagittal plane of the shaft; and
acquiring 3-D ultrasound data with the rotating transducer array.

19. The method of claim 18, wherein the transducer array includes a single transducer array disposed along a center-line of the shaft.

20. The method of claim 18, wherein the transducer array includes a first transducer array disposed along a center-line of the shaft and a second transducer array disposed transverse to the center-line of the shaft.
